(19) United States
(12) Patent Application Publication
(10) Pub. No.: US 2011/0263879 A1
(43) Pub. Date: Oct. 27, 2011

(54) ZWITTERIONIC PHOSPHONIUM SALTS

(75) Inventors: Tak-Hang Chan, Toronto (CA); Congde Huo, Lanzhou (CN); Xun He, Hockessin, DE (US)

(73) Assignee: THE HONG KONG POLYTECHNIC UNIVERSITY, Hung Hom, Kowloon (HK)

(21) Appl. No.: 13/056,561

(22) PCT Filed: Jul. 29, 2009
(86) PCT No.: PCT/CA09/01074

§ 371 Date: Jul. 13, 2011

Related U.S. Application Data
(60) Provisional application No. 61/084,360, filed on Jul. 29, 2008.

(51) Int. Cl. C07F 9/54; C07C 41/58; C07D 317/28; C07C 1/20; C07C 201/12; C07C 41/30; C07C 17/26; C07C 45/68; C07C 41/56; C07C 67/44

(52) U.S. Cl. 549/451; 562/35; 560/14; 568/592; 568/594; 560/55; 560/51; 560/104; 568/928; 568/631; 570/200; 568/312; 585/469

(57) ABSTRACT

A zwitterionic phosphonium salt of Formula I: wherein n is 0 or 1; R is H or SO₃; R′ is selected from the group consisting of C₁-C₁₀ alkyl, C₂-C₁₀ alkenyl, C₂-C₁₀ alkynyl, C₃-C₁₀ cycloalkyl, phenyl, substituted phenyl, benzyl and C₁-C₁₀ alkoxy-carbonyl; R′ is CX₃ when n is O; and X is selected from the group consisting of F, Cl, Br and I. The zwitterionic phosphonium salts are useful reagents for the preparation of alkenes and acetals from the corresponding aldehyde.

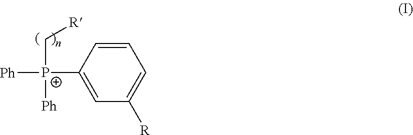

### CROSS REFERENCE TO RELATED APPLICATIONS

The present application claims the benefit of U.S. Provisional Application No. 61/084,360 filed Jul. 29, 2008, the entire contents of which are incorporated by reference.

### FIELD

The present disclosure broadly relates to zwitterionic phosphonium salts. More specifically, but not exclusively, the present disclosure relates to zwitterionic phosphonium sulfonates as well as to a process for their preparation.

### BACKGROUND

In the past few decades, considerable effort has been devoted toward the development of organocatalysts and supports to bind catalysts, reagents, or scavengers in order to facilitate the purification process following chemical reaction.

Following the introduction of polystyrene resins by Merrifield for peptide synthesis, insoluble solid polymer resins have also been adopted as supports for reagents and catalysts [1]. It is recognized however that these immobilized systems often react more slowly than their solution phase counterparts [2]. To overcome these limitations, efforts have been directed toward the development of soluble polymers [3] such as poly-(ethylene glycol) (PEG) [4] and non-cross-linked polystyrene (NCLP) [5] or fluorous phase synthesis [6] to restore homogenous reaction conditions. In these cases, the phase separation depends on the difference in the molecular weight of the support and the product or on the affinity of the fluorous tag for fluorous solvents.

Recently, the use of ion tags as soluble supports for organic synthesis has been explored [7]. Phase separation depends on the differential solubility of the ionic moiety in polar versus non-polar solvents.

The Wittig reaction is an important reaction in organic synthesis. However the separation of the alkene product from the reaction by-product triphenylphosphine oxide (Ph₃PO) is a classical problem that typically requires tedious chromatography or recrystallization. To overcome this problem, polymer bound [8] or fluorous-tagged [9] phosphines have been developed.

Organocatalytic reactions are of considerable interest in chemical processes [10]. Relative to the metal-based catalysts, organocatalysts avoid the use of metals which, in many instances, may be expensive, corrosive or toxic. Furthermore, organocatalysts can be chemically altered to confer unique properties such as reaction selectivity. While most metal catalysts function as Lewis acids, organocatalysts tend to function as either Lewis bases [11] or as Brønsted acids [12]. Metal-free Lewis acid organocatalysts are relatively rare and most of them are silicon based [13]. Recently, phosphonium salts have been advanced as metal-free Lewis acid catalysts by virtue of the hypervalent interaction through the formation of pentacoordinate intermediates [14]. Examination of a series of phosphonium salts as catalysts for a Diels-Alder reaction led to the conclusion that the formation of a five-membered dioxaphosphacycle appeared to play role for the salts to efficiently function as Lewis acid catalysts.

The present disclosure refers to a number of documents, the content of which is herein incorporated by reference in their entirety.

### SUMMARY

The present disclosure relates to zwitterionic phosphonium salts.

As broadly claimed, the present invention relates to zwitterionic phosphonium sulfonates as well as to a process for their preparation.

In an embodiment, the present disclosure relates to zwitterionic phosphonium sulfonates useful as versatile reagents in chemical synthesis. In a further embodiment, the present disclosure relates to zwitterionic phosphonium sulfonates useful as Wittig reagents for the preparation of alkenes. In a further embodiment, the present disclosure relates to zwitterionic phosphonium sulfonates useful as reagents for the preparation of acetals. In yet a further embodiment, the present disclosure relates to a method for preparing alkenes using zwitterionic phosphonium sulfonates. In yet a further embodiment, the present disclosure relates to a method for preparing acetals using zwitterionic phosphonium sulfonates. In yet a further embodiment, the present disclosure relates to zwitterionic phosphonium sulfonates that are recoverable following their use as reagents in chemical synthesis.

In an embodiment, the present disclosure relates to a zwitterionic phosphonium salt of Formula I:

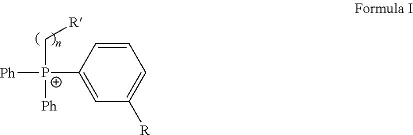

wherein:

R is H or SO₃⁻;

n is 0 or 1;

R is H or SO₃⁻;

R′ is selected from the group consisting of C₁-C₁₀ alkyl, C₂-C₁₀ alkenyl, C₂-C₁₀ alkynyl, C₃-C₁₀ cycloalkyl, phenyl, substituted phenyl, benzyl and C₁-C₁₀ alkoxycarbonyl;

R′ is CX₃ when n is 0; and

X is selected from the group consisting of F, Cl, Br and I.

In an embodiment, the present disclosure relates to a zwitterionic phosphonium salt having formula:

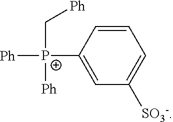

In an embodiment, the present disclosure relates to a zwitterionic phosphonium salt having formula:

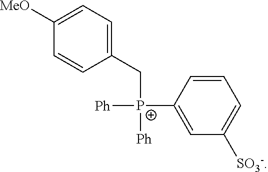

In an embodiment, the present disclosure relates to a zwitterionic phosphonium salt having formula:

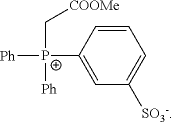

In an embodiment, the present disclosure relates to a zwitterionic phosphonium salt having formula:

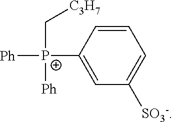

In an embodiment, the present disclosure relates to a zwitterionic phosphonium salt having formula:

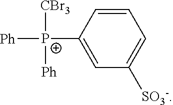

In an embodiment, the present disclosure relates to a method for converting an aldehyde functionality into an alkene functionality, the method comprising reacting a substrate bearing an aldehyde function with a zwitterionic phosphonium salt of Formula I:

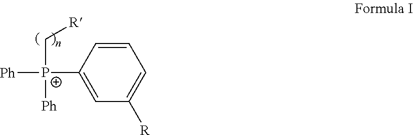

wherein:

n is 1;

R is H or SO₃⁻; and

R′ is selected from the group consisting of C₁-C₁₀ alkyl, C₂-C₁₀ alkenyl, C₂-C₁₀ alkynyl, C₃-C₁₀ cycloalkyl, phenyl, substituted phenyl, benzyl and C₁-C₁₀ alkoxycarbonyl;

in the presence of a base.

In an embodiment, the present disclosure relates to a method for converting an aldehyde functionality into an acetal functionality, the method comprising the step of reacting a substrate bearing an aldehyde function with a zwitterionic phosphonium salt of Formula I:

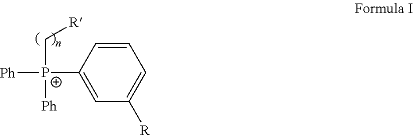

wherein:

n is 0 or 1;

R is H or SO₃⁻;

R′ is a C₁-C₁₀ alkoxycarbonyl;

R′ is CX₃ when n is 0; and

X is selected from the group consisting of F, Cl, Br and I;

in the presence of an alcohol.

In an embodiment, the present disclosure relates to a kit comprising at least one phosphonium salt of Formula I:

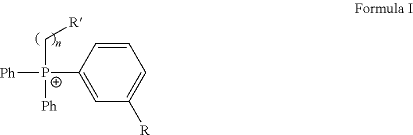

wherein:

R is H or SO₃⁻;

n is 0 or 1;

R is H or SO₃⁻;

R′ is selected from the group consisting of C₁-C₁₀ alkyl, C₂-C₁₀ alkenyl, C₂-C₁₀ alkynyl, C₃-C₁₀ cycloalkyl, phenyl, substituted phenyl, benzyl and C₁-C₁₀ alkoxycarbonyl;

R′ is CX₃ when n is 0; and

X is selected from the group consisting of F, Cl, Br and I.

The foregoing and other objects, advantages and features of the present disclosure will become more apparent upon reading of the following non-restrictive description of illustrative embodiments thereof, given by way of example only.

### DETAILED DESCRIPTION OF ILLUSTRATIVE EMBODIMENTS

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this specification pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The present description refers to a number of chemical terms and abbreviations used by those skilled in the art. Nevertheless, definitions of selected terms are provided for clarity and consistency.

Abbreviations: NMR: Nuclear Magnetic Resonance; MS: Mass Spectrometry; m.p.: melting point; HRMS: High Resolution Mass Spectrometry; ESI: Electrospray Ionization; FAB: Fast Atom Bombardment; TLC: Thin Layer Chromatography; FCC: Flash Column Chromatography; SPE: Solid-Phase Extraction; EtOAc: Ethyl Acetate; CH₂Cl₂: Dichloromethane; CDCl₃: Chloroform-d; DMAP: 4-(N,N-dimethylamino)pyridine; TFA: Trifluoroacetic Acid; AcOH: Acetic Acid; TPPMS: Triphenylphosphine-m-Sulfonate; TPPMSO: Triphenylphosphine-m-Sulfonate Oxide; TMSCl: Trimethylsilyl chloride; TMSOTf: Trimethylsilyl trifluoromethanesulfonate; TMSOFs: Trimethylsilylfluorosulfonate; Ph: Phenyl; LiAlH₄: Lithium Aluminum Hydride; LiHMDS: Lithium hexamethyldisilazide; SiHCl₃: Trichloro Silane; PhCN: Phenylnitrile: Bzl: Benzyl; NEt₃: Triethylamine; PhNMe₂: N,N-Dimethyl Phenylamine; CBr₄ Carbon Terabromide; MgSO₄: Magenium Sulfate; PTSA: p-Toluene Sufonic Acid; PEG: Polyethylene Glycol; DMF: Dimethyl Formamide; DMSO: Dimethyl Sulfoxide; and THF: Tetrahydrofuran.

As used herein, the term “alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of alkyl residues containing from 1 to 18 carbon atoms are methyl, ethyl, propyl, butyl, pentyl, hexyl, heptyl, octyl, nonyl, decyl, undecyl, dodecyl, tetradecyl, hexadecyl and octadecyl, the n-isomers of all these residues, isopropyl, isobutyl, isopentyl, neopentyl, isohexyl, isodecyl, 3-methylpentyl, 2,3,4-trimethylhexyl, sec-butyl, tert-butyl, or tert-pentyl. A specific group of alkyl residues is formed by the residues methyl, ethyl, n-propyl, isopropyl, n-butyl, isobutyl, sec-butyl and tert-butyl.

As used herein, the term “lower alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of lower alkyl residues containing from 1 to 6 carbon atoms are methyl, ethyl, propyl, isopropyl, butyl, isobutyl, tert-butyl, pentyl, isopentyl, neopentyl, and hexyl.

As used herein, the term “alkylene” can be a linear saturated divalent hydrocarbon radical of one to six carbon atoms or a branched saturated divalent hydrocarbon radical of three to six carbon atoms. Examples of alkylene residues are methylene, ethylene, 2,2-dimethylethylene, propylene, 2-methylpropylene, butylene, and pentylene.

In an embodiment of the present disclosure, the alkyl and alkylene groups may be substituted by replacing one or more hydrogen atoms by alternative non-hydrogen groups. These include, but are not limited to, halo, hydroxy, alkyloxy, and amino.

As used herein, the term “alkenyl” can be straight-chain or branched unsaturated alkyl residues that contain one or more, for example one, two or three double bonds which can be in any suitable position. Of course, an unsaturated alkyl residue has to contain at least two carbon atoms. Examples of unsaturated alkyl residues are alkenyl residues such as vinyl, 1-propenyl, allyl, butenyl or 3-methyl-2-butenyl.

As used herein the term “alkynyl” can be straight-chain or branched unsaturated alkyl residues that contain one or more, for example one, two or three, triple bonds which can be in any suitable position. Of course, an unsaturated alkyl residue has to contain at least two carbon atoms. Examples of unsaturated alkyl residues are alkynyl residues such as ethynyl, 1-propynyl or propargyl.

As used herein, the term “cycloalkyl” can be monocyclic or polycyclic, for example monocyclic, bicyclic or tricyclic, i.e., they can for example be monocycloalkyl residues, bicycloalkyl residues and tricycloalkyl residues, provided they have a suitable number of carbon atoms and the parent hydrocarbon systems are stable. A bicyclic or tricyclic cycloalkyl residue has to contain at least 4 carbon atoms. In an embodiment, a bicyclic or tricyclic cycloalkyl residue contains at least 5 carbon atoms. In a further embodiment, a bicyclic or tricyclic cycloalkyl residue contains at least 6 carbon atoms and up to the number of carbon atoms specified in the respective definition. Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C₁-C₄ alkyl. Examples of substituted cycloalkyl residues are 4-methylcyclohexyl and 2,3-dimethylcyclopentyl. Examples of parent structures of bicyclic ring systems are norbornane, bicyclo[2.2.1]heptane, bicyclo[2.2.2]octane and bicyclo[3.2.1]octane.

As used herein, the term “aryl” means an aromatic substituent which is a single ring or multiple rings fused together. When formed of multiple rings, at least one of the constituent rings is aromatic. In an embodiment, aryl substituents include phenyl and naphthyl groups.

As used herein, the term “substituted phenyl” is understood as being phenyl having a substituent selected from the group consisting of amino, —NH(lower alkyl), and —N(lower alkyl)₂, as well as being mono-, di- and tri-substituted phenyl comprising substituents selected from the group consisting of lower alkyl, methoxy, methylthio, halo, cyano, hydroxy, amino, NH(lower alkyl), and —N(lower alkyl)₂.

The term “heteroaryl”, as used herein, is understood as being unsaturated rings of five or six atoms containing one or two O- and/or S-atoms and/or one to four N-atoms, provided that the total number of hetero-atoms in the ring is 4 or less. The heteroaryl ring is attached by way of an available carbon or nitrogen atom. Non-limiting examples of heteroaryl groups include 2-, 3-, or 4-pyridyl, 4-imidazolyl, 4-thiazolyl, 2- and 3-thienyl, and 2- and 3-furyl. The term “heteroaryl”, as used herein, is understood as also including bicyclic rings wherein the five or six membered ring containing O, S and N-atoms as defined above is fused to a benzene or pyridyl ring. Non-limiting examples of bicyclic rings include but are not limited to 2- and 3-indolyl as well as 4- and 5-quinolinyl.

The invention contemplates that for any stereocenter or axis of chirality for which the stereochemistry has not been defined, that stereocenter or axis of chirality can be present in its R form, S form, or as a mixture of the R and S forms, including racemic and non-racemic mixtures.

As used herein, the term “heteroatom” refers to oxygen, sulfur or nitrogen.

As used herein, the term “halogen” or “halo” refers to fluorine, chlorine, bromine, iodine, and fluoro, chloro, bromo and iodo.

Formation of Alkenes Using Zwitterionic Phosphonium Sulfonates

Because the sodium salt of triphenylphosphine-m-sulfonate (1) is commercially available [15], the ionic salt 1,2-dimethyl-3-butylimidazolium triphenylphosphine-m-sulfonate (2) was prepared from the reaction of 1 with 1,2-dimethyl-3-butylimidazolium bromide (Scheme 1). Reaction of 2 with benzyl tosylate yielded the zwitterionic phosphonium salt 3a together with 1,2-dimethyl-3-butylimidazolium tosylate. Alternatively, the zwitterionic phosphonium salt 3a can be prepared from the reaction of 1 with benzyl bromide (Scheme 1). Zwitterionic phosphonium sulfonate salts 3b-d were prepared similarly from the corresponding bromides.

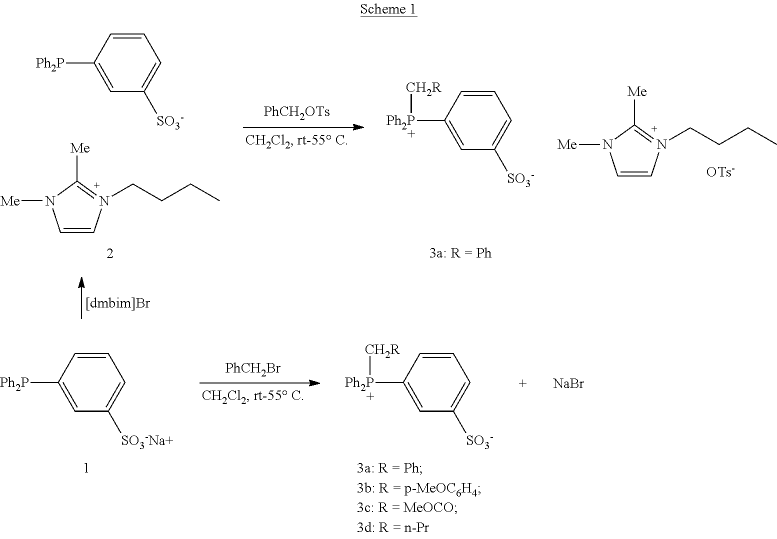

The Wittig reaction of 3 with various carbonyl compounds was evaluated in different base/solvent conditions (Scheme 2) and the results summarized in Table 1.

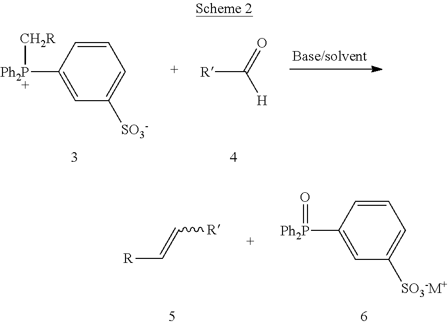

While NaOH/H₂O proved to be efficient to effect the reaction between 3a and p-nitrobenzaldehyde (4a) in good yield, NaOH/MeOH was generally more effective for all the aldehydes tested. The separation of the product alkene 5 from the by-product phosphine oxide 6 proved to be unexpectedly easy. After the reaction was completed, a less polar solvent, a non-limiting example of which includes diethyl ether, was added to the reaction mixture to allow precipitation of the phosphine oxide by-product 6. Following filtration, the organic layer was free of 3a and 6, as evident from TLC and ³¹P NMR. As evident from ¹H NMR analysis, the product alkene 5 generally required no further purification. Trans-cinnamaldehyde 4f and hydrocinnamaldehyde 4g were readily converted to the corresponding diene 5f and alkene 5g. Unexpectedly, no reaction could be observed between 3a and ketones such as benzophenone (4h), acetophenone, cyclohexanone or acetone in NaOH/MeOH, the ketones being quantitatively recovered. The reaction therefore appears to be chemoselective for aldehydes. Thus, 4-acetylbenzaldehyde (4i) reacted chemoselectively with 3a to provide compound 5i in substantially quantitative yield.

TABLE 1
Wittig Reaction of 3a with various aldehydes.

| Entry | Aldehyde (RCHO) | Product | Base/Solvent | Yield (%) (E:Z) |
|---|---|---|---|---|
| 1 | 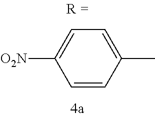 | 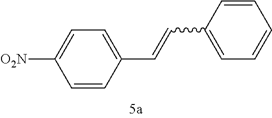 | NaOH/MeOH NaOH/H₂O LiHMDS/THF LiHMDS/DCM K₂CO₃/MeOH K₂CO₃/iPrOH | >95 (1.1:1.0) >95 (1.6:1.0) 91 (2.4:1.0) 88 (1.8:1.0) 83 (1.2:1.0) 0 |
| 2 | 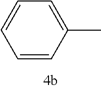 | 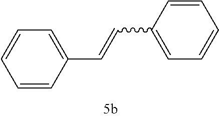 | NaOH/MeOH NaOH/H₂O | >95 (1.7:1.0) 83 (3.6:1.0) |
| 3 | 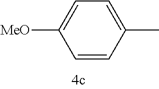 | 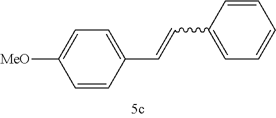 | NaOH/MeOH NaOH/H₂O | 83 (1.1:1.0) trace |
| 4 | 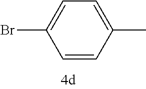 | 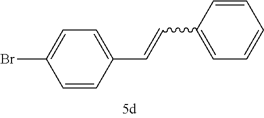 | NaOH/MeOH | >95 (1.2:1.0) |
| 5 | 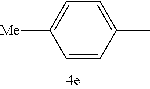 | 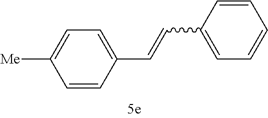 | NaOH/MeOH | >95 (2.0:1.0) |
| 6 | 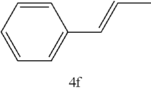 | 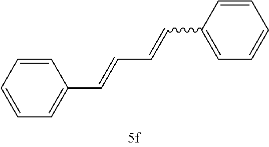 | NaOH/MeOH | >95 (2.3:1.0) |
| 7 | 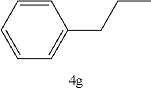 | 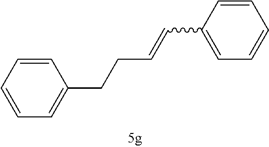 | NaOH/MeOH | 78 (1.2:1.0) |
| 8 | Benzophenone 4h | 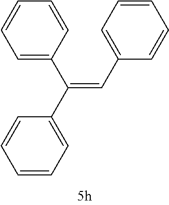 | LiHMDS/THF NaOH/MeOH | 20 0 |
| 9 | 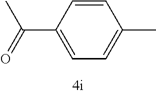 | 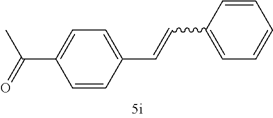 | NaOH/MeOH | >95 (1.1:1.0) |

Finally, reaction of compound 3b with 3,5-dimethoxybenzaldehyde in NaOH/MeOH provided methylated resveratrol 5j in good yield (Scheme 3). Compound 5j can subsequently be readily converted to resveratrol [16].

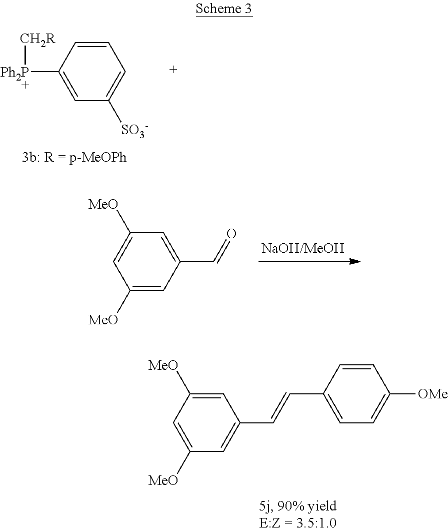

Using the more acidic zwitterionic phosphonium salt 3c, potassium carbonate could be used as the base to effect the Wittig reaction. As shown hereinbelow in Table 2, various aromatic and aliphatic aldehydes 4 were converted to their corresponding alkenes 5 in good yields.

TABLE 2
Wittig Reaction of 3c with various aldehydes.

| Entry | Aldehyde (RCHO) | Product | Base/Solvent | Yield (%) (E:Z) | |
|---|---|---|---|---|---|
| 1 | 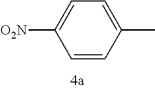 | 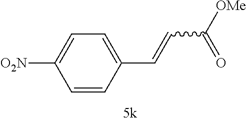 | K₂CO₃/MeOH | >95 | (2.6:1.0) |
| 2 | 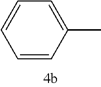 | 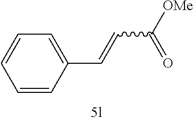 | K₂CO₃/MeOH | >95 | (2.8:1.0) |
| 3 | 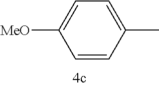 | 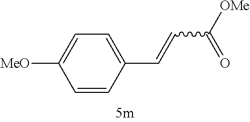 | K₂CO₃/MeOH | >95 | (2.6:1.0) |
| 4 | 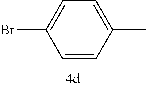 | 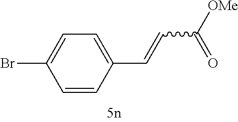 | K₂CO₃/MeOH | >95 | (2.6:1.0) |
| 5 | 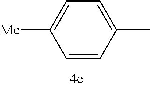 | 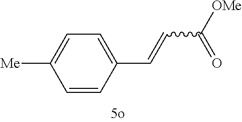 | K₂CO₃/MeOH | >95 | (3.2:1.0) |
| 6 | 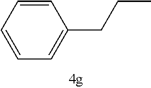 | 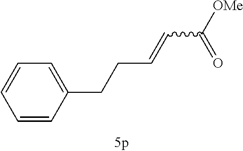 | K₂CO₃/MeOH | >95 | (1.7:1.0) |
| 7 | 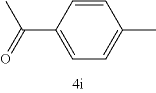 | 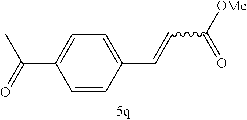 | K₂CO₃/MeOH | >95 | (2.7:1.0) |

As was previously observed, ketones such as benzophenone, acetophenone, cyclohexanone and acetone, were found to be unreactive under the reaction conditions and were quantitatively recovered. 4-Acetylbenzaldehyde (4i) reacted chemoselectively with 3c to provide compound 5q in substantially quantitative yield. Separation of the product alkene from the reaction mixture could again be conveniently achieved by the addition of a less polar solvent, a non-limiting example of which includes diethyl ether, to allow precipitation of the phosphine oxide by-product 6.

In an embodiment of the present disclosure, the zwitterionic phosphonium salt 3c is generated in situ. Mixing triphenylphosphine-m-sulfonate (1), methyl bromoacetate, potassium carbonate and aldehyde 4 in methanol, followed by stirring at room temperature, yielded the desired α,β-unsaturated ester 5 in good yield and high purity as confirmed by ¹H NMR analysis. This “one-pot” reaction provides a more convenient alternative over the Horner-Wadsworth-Emmons (HWE) modification [17] to effect the olefination of aldehydes. However, the HWE reaction remains the more stereoselective alternative, affording the thermodynamically more stable (E)-α,β-unsaturated esters. The mixture of stereoisomers obtained using the zwitterionic phosphonium sulfonates of the present invention can be conveniently isomerized to the thermodynamically more stable E-isomer [18]. Following the “one-pot” reaction of triphenylphosphine-m-sulfonate (1), methyl bromoacetate, potassium carbonate and benzaldehyde 4b in methanol, the reaction side-product phosphine oxide 6 was precipitated and removed by filtration. The crude reaction product was subsequently dissolved THF followed by the addition of 25 mol % diphenyl disulfide. After overnight refluxing, pure E-5l was obtained.

Using the less acidic zwitterionic phosphonium salt 3d, a stronger base was used to effect the Wittig reaction. In an embodiment of the present disclosure, LiHMDS in THF was reacted with 3d and nitrobenzaldehyde (4a) to afford 1-(4-nitrophenyl)pent-1-ene in 90% isolated yield (E:Z=2.1:1.0). The isolation of the alkene product from the reaction by-product phosphine oxide 6 was again achieved by ether precipitation.

Conversion or Recycling of TPPMSO (6) to TPPMS (1)

TPPMSO was conveniently reconverted into TPPMS using SiHCl₃/PPh₃ [19]. The reaction mixture was quenched using a NaOH solution followed by the addition of methanol. The solid silica gel derived from the hydrolysis of the chlorosilanes was removed by filtration. The filtrate was subsequently concentrated and washed with ether. The desired TPPMS was obtained as a white solid.

Acetalization Using Zwitterionic Phosphonium Sulfonates

Acetalization reactions are typically affected and catalyzed using Brønsted acids such as HCl and PTSA, or metal-based Lewis acid such as TiCl₄, ZrCl₄, Sc(OTf)₃, LaCl₃, CeCl₃, InCl₃, RuCl₃, Bi(OTf)₃ and MeReO₃, or silicon-based Lewis acids such as TMSCl, TMSOTf and TMSOFs [20]. It has been unexpectedly discovered that by introducing an electron withdrawing group into triphenylphosphine-m-sulfonate (1), a zwitterionic phosphonium sulfonate salt (9g and 9h) is generated which constitutes a useful reagent for the preparation of acetals from the corresponding aldehydes. Non limiting examples of suitable electron withdrawing groups comprise CF₃, CCl₃, CBr₃ and CI₃. In light of the present disclosure, it is well within one of ordinary skill in the art to determine further electron withdrawing groups without departing from the spirit, scope and nature of the present disclosure. The presence of the electron withdrawing group on the triphenylphosphine-m-sulfonate (1) facilitates the activation of the aldehyde (Lewis base) by the sulfonate salt. A series of phosphonium salts 9 were prepared and tested for their efficiency for the catalytic acetalization of p-nitrobenzaldehyde (10a) and the results summarized in Table 3. The catalytic acetalization reactions were performed in methanol at 25° C. over a period of 12 hours using 5 mol % of the phosphonium sulfonate salt (Scheme 4).

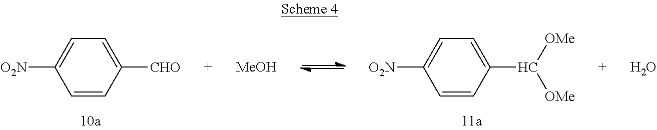

As expected, phosphonium salts 9a and 9b did not provide any of the desired acetal product. However, phosphonium salt 9c, bearing an electron-withdrawing ester moiety, afforded the acetal product 11a in good yield (87%). Phosphonium salt 9d, comprising the more electron-withdrawing CBr₃ group, afforded the acetal product in slightly improved yield (90%). The introduction of an electron withdrawing group in the form of a sulfonate on one of the phenyl rings did not improve the reactivity of the phosphonium salts as no reaction could be observed for compounds 9e and 9f. Compound 9g was only poorly soluble in methanol and only 15% of the desired acetal product was observed after 12 hours. Surprisingly, compound 9h, readily prepared by the reaction of triphenylphosphine-m-sulfonate (1) with CBr₄, showed greater catalytic activity than 9d, affording the acetal product 11a in substantially quantitative yield (>95%).

TABLE 3
Catalytic acetalization of p-nitrobenzaldehyde using
zwitterionic phosphonium salts 9.

| | | | % Yield |
|---|---|---|---|
| Entry | Phosphonium salt 9 used | | of 11a |
| 1 | 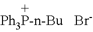 | 9a | 4 |
| 2 | 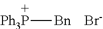 | 9b | 0 |
| 3 | 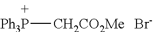 | 9c | 87 |
| 4 | 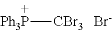 | 9d | 90 |
| 5 | 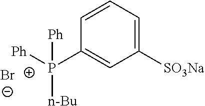 | 9e | trace |
| 6 | 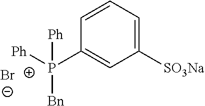 | 9f | 0 |
| 7 | 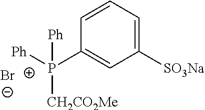 | 9g | —^{a} |
| 8 | 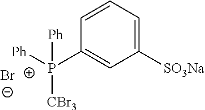 | 9h | >95% |
| 9 | 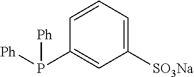 | (TPPMS) | 0 |
| 10 | CBr₄ | | 0 |
| 11 | PTSA | | 67 |

^{a}Compound 9g was not completedly dissolved in the reaction mixture and 15% of 11a was observed at the end of 12 hrs.

As illustrated hereinbelow in Table 4, the phosphonium sulfonate salt 9h effectively catalyzed the acetalization of both aromatic and aliphatic aldehydes using methanol. In all cases, the phosphonium sulfonate salt 9h provided superior results over phosphonium salt 9d (comparison of entries 2, 5 and 9 with entries 1, 4 and 8 respectively) which appears indicative of an additional effect imparted by the sulfonate group. In the case of p-methoxybenzaldehyde, the lower yield (entry 8) obtained with 9h was likely due to the equilibrium being adversely affected by the methoxy substituent. Indeed, by adding a dehydrating agent (e.g. MgSO₄) to the reaction mixture, a substantially higher yield (77%) of the acetal product could be obtained.

TABLE 4
Catalytic acetalization of various aldehydes using
zwitterionic phosphonium sulfonate 9h.

| Entry | Aldehyde 10 | Catalyst 9 used | % Yield of 11 |
|---|---|---|---|
| 1 | 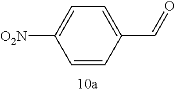 | 9h | >95 |
| 2 | 10a | 9d | 90 |
| 3 | 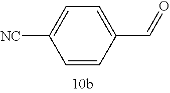 | 9h | 91 |
| 4 | 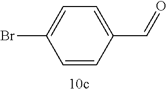 | 9h | 93 |
| 5 | 10c | 9d | 30 |
| 6 | 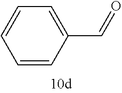 | 9h | >95 |
| 7 | 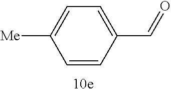 | 9h | 78 |
| 8 | 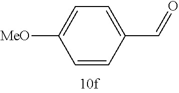 | 9h | 47(77) |
| 9 | 10f | 9d | 0 |
| 10 | 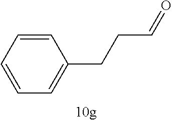 | 9h | >95 |
| 11 | 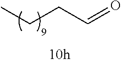 | 9h | >95 |

Zwitterionic phosphonium sulfonate 9h also effected the acetalization of p-nitrobenzaldehyde using a variety of alcohols as summarized hereinbelow in Table 5. In the case of higher boiling alcohols, a stoichiometric amount of the alcohol was used and the acetalization reaction was carried out in CH₂Cl₂ as the solvent.

TABLE 5
Acetalization of p-nitrobenzaldehyde using zwitterionic
phosphonium sulfonate 9h (5 mol %) and a variety of alcohols

| Entry | Alcohol used | Acetal formed | % Yield |
|---|---|---|---|
| 1 | MeOH | 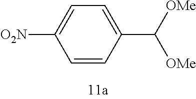 | >95 |
| 2 | EtOH | 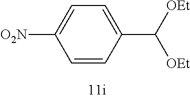 | >95 |
| 3 | ⁱPrOH (in DCM) | 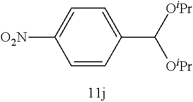 | 91 |
| 4 | BnOH (in DCM) | 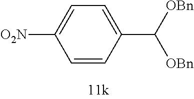 | >95 |
| 5 | (CH₂OH)₂ (in DCM) | 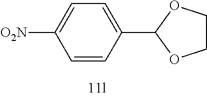 | >95 |

The reaction conditions for the acetalization reactions of the present disclosure were remarkably mild, relative to the high reaction temperatures and long reaction times usually required for acetalization reactions mediated by Brønsted acids such as HCl and PTSA [21]. As was previously observed, no reaction could be observed between 9h and ketones such as benzophenone, acetophenone, cyclohexanone and acetone, the ketones being quantitatively recovered. The reaction therefore again appears to be chemoselective for aldehydes. Thus, 4-acetylbenzaldehyde (4i) reacted chemoselectively with 9h (5 mol %) and methanol to provide the corresponding acetal in substantially quantitative yield. Due to the zwitterionic nature of 9h, the catalyst is soluble in relatively polar organic solvents such as methanol and can thus be readily and quantitatively recovered from the reaction mixture by the addition of a non-polar organic solvent such as ether. Therefore, as was previously observed for the formation of alkenes, the separation and recovery of 9h from the reaction mixture was effectively carried out by precipitation using a non-polar solvent (e.g. ether) following completion of the reaction. Finally, recovered 9h can be reused without loss of catalytic activity. In fact, using the acetalization of p-nitrobenzaldehyde with methanol as a model system, 9h was used in seven cycles of acetalization without diminished yield.

### EXPERIMENTAL

All reagents were obtained commercially and used as received unless otherwise specified. TLC inspections were performed on silica gel GF254 plates. NMR spectra were recorded at 400 MHz (¹H NMR), 100 MHz (¹³C NMR) and 81 MHz (³¹P NMR) at room temperature in CDCl₃, DMSO-d₆ and CD₃OD respectively.

### Example 1

Typical Procedure for the Preparation of Phosphonium Salts 3a-d

A mixture of triphenylphosphine-m-sulfonate (1) (728 mg, 2 mmol) and a slight excess of the corresponding bromide reagent (2.4 mmol) were stirred overnight at 50° C. Ether was added and the precipitate was filtered to afford the target phosphonium sulfonate salts as white solids.

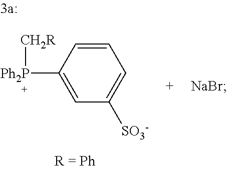

¹H NMR (400 MHz, d₆-DMSO): δ 8.05 (d, J=7.2 Hz, 1H), 7.91-7.83 (m, 3H), 7.76-7.58 (m, 10H), 7.28-7.19 (m, 3H), 6.96 (d, J=7.2 Hz, 2H), 5.19 (d, J=16 Hz, 2H). ³¹P NMR (81 MHz, DMSO-d₆): δ 23.3 (s). HRMS m/z calculated for C₂₅H₂₂PO₃S⁺ 433.1022, found 433.1025.

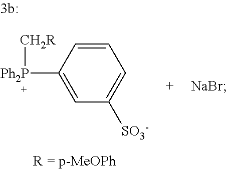

¹H NMR (400 MHz, DMSO-d₆): δ 8.05 (d, J=7.2 Hz, 1H), 7.91-7.60 (m, 13H), 6.88 (d, J=7.2 Hz, 2H), 6.78 (d, J=7.2 Hz, 2H), 5.11 (d, J=14.8 Hz, 2H), 3.67 (s, 3H). ³¹P NMR (81 MHz, DMSO-d₆): δ 23.7 (s). HRMS m/z calculated for C₂₆H₂₄PO₄S⁺ 463.1127, found 463.1125.

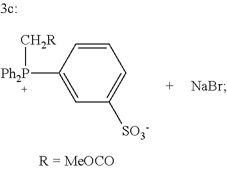

¹H NMR (400 MHz, DMSO-d₆): δ 8.06-7.72 (m, 14H), 5.40 (d, J=14.4 Hz, 2H), 3.59 (s, 3H). ³¹P NMR (81 MHz, DMSO-d₆): δ 25.4 (s). HRMS m/z calculated for C₂₁H₂₀PO₅S⁺ 433.0764, found 433.0767.

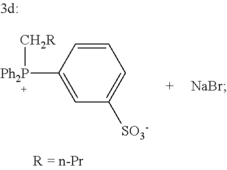

¹H NMR (400 MHz, DMSO-d₆): δ 8.05 (d, J=7.6 Hz, 1H), 7.91-7.73 (m, 13H), 3.06 (m, 2H), 1.47 (m, 4H), 0.87 (t, J=6.4 Hz, 3H). ³¹P NMR (81 MHz, DMSO-d₆): δ 23.3 (s). HRMS m/z calculated for C₂₂H₂₄PO₃S⁺ 399.1178, found 399.1181.

### Example 2

Typical Procedure for the Formation of Alkenes Using Phosphonium Sulfonate Salts 3a and 3b

NaOH (0.25 mmol) was added to phosphonium salt 3a or 3b (0.2 mmol) suspended in methanol (1 mL) The reaction mixture was subsequently stirred over a period of 5 minutes followed by the addition of an aldehyde 4 (0.2 mmol) substrate. The reaction mixture was stirred at room temperature overnight. The phosphine oxide by-product 6 was precipitated by the addition of ether (3 mL). The reaction mixture was finally filtered and the filtrate evaporated to yield the alkene product 5. Alkene products 5a-5j are known compounds whose characterization was found to be in agreement with the literature reports.

### Example 3

Typical Procedure for the Formation of Alkenes Using Phosphonium Sulfonate Salt 3c

Method 1: K₂CO₃ (0.25 mmol) was added to phosphonium salt 3c (0.2 mmol) suspended in methanol (1 mL). The reaction mixture was subsequently stirred over a period of 5 minutes followed by the addition of an aldehyde 4 (0.2 mmol) substrate. The reaction mixture was stirred at room temperature overnight. The phosphine oxide by-product 6 was precipitated by the addition of ether (3 mL). The reaction mixture was finally filtered and the filtrate evaporated to yield the alkene product 5.

Method 2: Triphenylphosphine-m-sulfonate (1) (73 mg, 0.2 mmol), methyl bromoacetate (31 mg, 0.2 mmol), K₂CO₃ (0.25 mmol) and an aldehyde 4 (0.2 mmol) substrate were dissolved in methanol (1 mL) and stirred at room temperature overnight. The phosphine oxide by-product 6 and any unreacted 1 were precipitated by the addition of ether (3 mL). The reaction mixture was finally filtered and the filtrate evaporated to yield the alkene product 5. Alkene products 5k-5q are known compounds whose characterization was found to be in agreement with the literature reports.

### Example 4

Typical Procedure for the Isomerization of a Mixture of E/Z Stereoisomers to Provide the more Thermodynamically Stable E-isomer.

A mixture of E- and Z-5l was prepared according to Method 2. Following the removal of the phosphine oxide by-product 6, the filtrate was concentrated and the crude reaction product dissolved in anhydrous THF (2 mL) followed by the addition of diphenyl disulfide (11 mg; 25 mol %). The reaction mixture was refluxed overnight under an argon atmosphere. NMR analysis confirmed the complete isomerization into the E-isomer. Pure E-5l was obtained following purification by chromatography.

### Example 5

Typical Procedure for the Formation of Alkenes Using Phosphonium Sulfonate Salt 3d

LiHMDS (0.2 mmol in THF) was added to phosphonium salt 3d (0.2 mmol) suspended in THF (1 mL). The reaction mixture was subsequently stirred over a period of 5 minutes followed by the addition of an aldehyde 4 (0.2 mmol) substrate. The reaction mixture was stirred at room temperature overnight. The phosphine oxide by-product 6 was precipitated by the addition of ether (3 mL) The reaction mixture was finally filtered and the filtrate evaporated to yield the alkene product 5.

### Example 6

General Procedure for the Conversion of TPPMSO(6) to TPPMS (1)

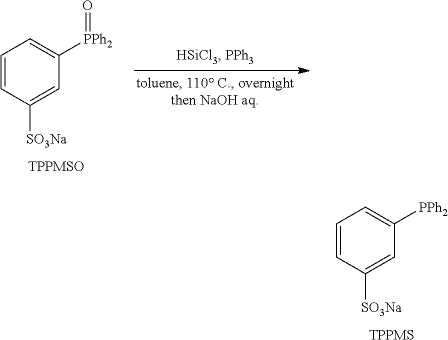

Phosphine oxide 6 (200 mg, 0.52 mmol) and triphenylphosphine (274 mg, 1.05 mmol) were suspended in toluene (10 mL) under an argon atmosphere using a 50 mL pressure tube. Trichlorosilane (1 mL, 10 mmol) was subsequently added to the mixture at room temperature. The reaction mixture was subsequently stirred at 110° C. overnight. After the mixture was cooled to ambient temperature, it was quenched with NaOH (2 mL, 20 wt %) followed by the subsequent addition of MeOH (25 mL). The reaction mixture was then filtered using a thin pad of celite. The filtrate was concentrated followed by the addition of fresh MeOH (25 mL). The solution was finally dried (Na₂SO₄) and concentrated under reduced pressure. The crude residue was washed with ether (3×2 mL) to afford TPPMS (1) as a white solid (170 mg, 90% yield). TPPMS (1): ¹H NMR (400 MHz, CD₃OD): δ 7.85-7.81 (m, 2H), 7.43-7.39 (m, 1H), 7.37-7.26 (m, 11H); ³¹P NMR (81 MHz, CD₃OD): δ-4.07 (s). TPPMSO (6): ¹H NMR (400 MHz, CDCl₃): δ 8.13-8.07 (m, 2H), 7.81-7.75 (m, 1H), 7.69-7.62 (m, 7H), 7.58-7.53 (m, 4H); ³¹P NMR (81 MHz, DMSO-d₆): 32.6 (s).

### Example 7

Typical Procedure for the Preparation of Phosphonium Salt 9h

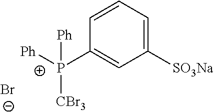

A mixture of TPPMS (1) (728 mg, 2 mmol) and carbon tetrabromide (663 mg, 2 mmol) was refluxed in methanol (10 mL) overnight. The reaction mixture was subsequently concentrated followed by the addition of ether (3×10 mL). The desired phosphonium salt 9h was obtained as white solid, 1 g (70% yield), m.p. 215° C. ¹H NMR (400 MHz, CD₃OD): δ 8.10-8.07 (m, 2H), 7.82-7.77 (m, 1H), 7.69-7.63 (m, 7H), 7.58-7.54 (m, 4H), ³¹P NMR (81 MHz, CD₃OD): δ 32.7 (s, 1P). ¹³C NMR (100 MHz, CD₃OD): δ 146.1, 146.0, 133.5, 133.4, 132.9, 132.8, 132.8, 132.0, 131.9, 131.8, 131.5, 130.4, 130.0, 129.9, 129.3, 129.2, 129.1, 129.0, 129.0, 128.9.

### Example 8

Typical Procedure for Acetal Formation Using Phosphonium Salts 9a-h

One of the phosphonium salts 9a-h (5 mol %) and an aldehyde 4 (0.2 mmol) substrate were dissolved in methanol (1 mL) and stirred at room temperature over a period of 12 hours. Ether (3 mL) was subsequently added and the reaction mixture filtered. The filtrate was subsequently concentrated to afford the desired acetal product. The procedure was repeated with TPPMS (1), CBr₄ and PTSA.

### Example 9

Typical Procedure for Acetal Formation Using Phosphonium Sulfonate Salt 9h and Various Aldehydes and Alcohols

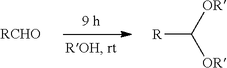

Phosphonium salt 9h (5 mol %) and an aldehyde 4 (0.2 mmol) substrate were dissolved in an alcohol solvent (1 mL in the case of MeOH and EtOH) or in DCM (1 mL; comprising a stoichiometric amount of the alcohol) and stirred at room temperature overnight. Ether (3 mL) was subsequently added and the reaction mixture filtered (98% recovery of 9h). The filtrate was subsequently concentrated to afford the desired acetal product.

### Example 10

Recycling Study of Phosphonium Sulfonate Salt 9h

Phosphonium salt 9h (5 mol %) and 4-nitrobenzaldehyde 4a (0.2 mmol) were dissolved in MeOH (1 mL) and stirred at room temperature overnight. Ether (3 mL) was subsequently added and the reaction mixture filtered. The recovered phosphonium salt 9h was redissolved in MeOH and reacted with further 4-nitrobenzaldehyde 4a. A total of seven (7) reaction cycles were performed, the yields of acetal product being respectively 99%, 98%, 96%, 97%, 96%, 97% and 97%.

It is to be understood that the disclosure is not limited in its application to the details of construction and parts as described hereinabove. The disclosure is capable of other embodiments and of being practiced in various ways. It is also understood that the phraseology or terminology used herein is for the purpose of description and not limitation. Hence, although the present disclosure has been described hereinabove by way of illustrative embodiments thereof, it can be modified without departing from the spirit, scope and nature as defined in the appended claims.

### REFERENCES

1. (a) Guillier, F.; Orain, D.; Bradley, M. Chem. Rev. 2000, 100, 2091; (b) James, I. W. Tetrahedron 1999, 55, 4855; (c) Ley, S. V.; Baxendale, I. R. Chem Rec. 2002, 2, 377.
2. Smith, C. D.; Baxendale, I. R.; Tranmer, G. K.; Baumann, M.; Smith, S. C.; Lwethwaite, R. A.; Ley, S. V. Org. Biomol. Chem. 2007, 5, 1562.
3. Bergbreiter, D. E. Chem. Rev. 2002, 102, 3345.
4. Toy, P. H.; Janda, K. D. Acc. Chem. Res. 2000, 33, 546.
5. Charette, A. B.; Boezio, A. A.; Janes, M. K. Org. Lett. 2000, 2, 3777.
6. (a) Horvath, I. T.; Rábai, J. Science 1994, 266, 72; (b) Studer, A.; Hadida, S.; Ferritto, S. Y.; Kim, P. Y.; Jeger, P.; Wipf, P.; Curran, D. P. Science 1997, 275, 823; (c) Zhang, W. Tetrahedron 2003, 59, 4475; Chem. Rev. 2004, 104, 2531.
7. (a) Fraga-Dubreuil, J.; Bazureau, J. P. Tetrahedron Lett. 2001, 42, 6097; (b) Miao, W.; Chan, T. H.; Org. Lett. 2003, 5, 5003; (c) Miao, W.; Chan, T. H. Acc. Chem. Res. 2006, 39, 897; (d) Poupon, J.-C.; Boezio, A. A.; Charette, A. B. Angew. Chem., Int. Ed. 2006, 45, 1415; (e) Stazi, F.; Marcoux, D.; Poupon, J.-C.; Latassa, D.; Charrette, A. B. Angew. Chem., Int. Ed. 2007, 46, 5011.
8. Bolli, M. H.; Ley, S. V. J. Chem. Soc. Perkin Trans. 1, 1998, 2243.
9. Galante, A.; Lhoste, P.; Sinou, D. Tetrahedron Letts. 2001, 42, 5425.
10. (a) Dalko, P. I.; Moisan, L. Angew. Chem. Int. Ed. 2004, 43, 5138; (b) de Figueriredo, R. M.; Christmann, M. Eur. J. Org. Chem. 2007, 2575.
11. (a) Denmark, S. E.; Beutner, G. L. Angew. Chem. Int. Ed. 2008, 47, 1560; (b) Orito, Y.; Nakajima, M. Synthesis, 2006, 1391.
12. (a) Akiyama, T.; Itoh, J.; Fuchibe, K. Adv. Synth. Catal. 2006, 348, 999; (b) Yamanaka, M.; Itoh, J.; Fuchibe, K.; Akiyama, T. J. Am. Chem. Soc., 2007, 129, 6756; (c) Zhou, W.; Xu, L.-W.; Yang, L.; Zhao, P.-Q.; Xia, C.-G. J. Mol. Catal. A., 2006, 249, 129.
13. (a) Prakash, G. K. S.; Panja, C.; Do, C.; Mathew, T.; Olah, G. A. Synlett, 2007, 2395; (b) Cai, S.; Yu, B. Org. Lett. 2003, 5, 3827.
14. (a) Terada, M.; Kouchi, M. Tetrahedron, 2006, 62, 401; (b) Wang, X.; Tian, S.-K. Tetrahedron Lett. 2007, 48, 6010.
15. (a) Cornils, B.; Kuntz, E. G. J. Organomet. Chem. 1995, 502, 177; (b) Baricelli, P. J.; Baricelli, D.; Lujano, E.; Melean, L. G.; Borusiak, M.; Lopez-Linares, F.; Bastidas, L. J.; Rosales, M. J. Mol. Catal. A 2007, 271, 180.
16. Ali, M. A.; Kondo, K.; Tsuda, Y. Chem. Pharm. Bull. 1992, 40, 1130-6.
17. Kurti, L.; Czako, B.; Editors Strategic Applications of Named Reactions in Organic Synthesis, 2005.
18. (a) Miyata, O.; Shinada, T.; Ninomiya, I.; Naito, T. Synthesis, 1990, 1123; (b) Baag, M.; Kar, A.; Argade, N. P. Tetrahedron 2003, 59, 6489; (c) Kim, I. S.; Dong, G. R.; Jung, Y. H. J. Org. Chem. 2007, 72, 5424.
19. Wu, H.-C.; Yu, J.-Q.; Spencer, J. B. Org. Lett. 2004, 6, 4675-4678.
20. (a) Chan, T. H.; Brook, M.; Chaly, T. Synthesis 1983, 203; (b) Vandewalle, M.; der Eycken, V.; Oppolzer, W.; Vullioud, C. Tetrahedron 1986, 42, 4035; (c) Lipshutz, B. H.; Burgres-Henry, J.; Roth, G. P. Tetrahedron Lett. 1993, 34, 995.
21. (a) Cameron, A. F. B.; Hunt, J. S.; Oughton, J. F.; Wilkinson, P. A.; Wilson, B. M. J. Chem. Soc. 1953, 3864; (b) Wenkert, E.; Goodwin, T. E. Synth. Commun. 1977, 7, 409.

1-9. (canceled)

10. A zwitterionic phosphonium salt of Formula I: 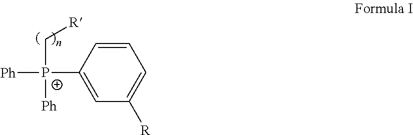
wherein:
n is 0 or 1;
R is SO₃⁻;
R′ is selected from the group consisting of C₁-C₁₀ alkyl, C₂-C_{10 alkenyl, C}₂-C₁₀ alkynyl, C₃-C₁₀ cycloalkyl, substituted phenyl, benzyl and C₁-C₁₀ alkoxycarbonyl when n is 1;
R′ is CX₃ when n is 0; and
X is selected from the group consisting of F, Cl, Br and I.

11. The zwitterionic phosphonium salt of claim 10 having formula:

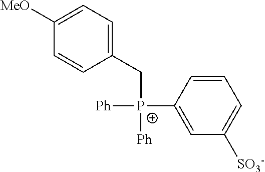

12. The zwitterionic phosphonium salt of claim 10 having formula:

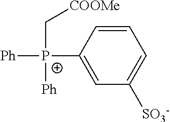

13. The zwitterionic phosphonium salt of claim 10 having formula:

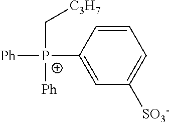

14. The zwitterionic phosphonium salt of claim 10 having formula:

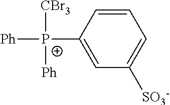

15. A method for converting an aldehyde functionality into an alkene functionality, the method comprising reacting a substrate bearing an aldehyde functionality with a zwitterionic phosphonium salt of Formula I: 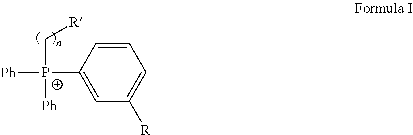
wherein:
n is 1;
R is SO₃⁻; and
R′ is selected from the group consisting of C₁-C₁₀ alkyl, C₂-C₁₀ alkenyl, C₂-C₁₀ alkynyl, C₃-C₁₀ cycloalkyl, phenyl, substituted phenyl, benzyl and C₁-C₁₀ alkoxycarbonyl;
in the presence of a base.

16. The method of claim 15, further comprising separating alkene product by adding to product mixture that includes a reaction solvent a different solvent in which phosphine oxide by-product is not soluble.

17. The method of claim 16, wherein the different solvent is diethyl ether.

18. The method of claim 16, further comprising separating phosphine oxide by-product precipitate via filtration.

19. The method of claim 18, wherein the alkene product is isolated by evaporation of the filtrate.

20. The method of claim 16, wherein the zwitterionic phosphonium salt of Formula I has the formula:

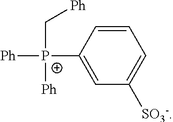

21. A method for converting an aldehyde functionality into an alkene functionality, the method comprising reacting a substrate comprising an aldehyde functionality with triphenylphosphine-meta-sulfonate, a compound comprising a halide or tosyl bound to a deprotonable carbon, and a base, in the presence of a solvent, to form a product mixture comprising an alkene product and a phosphine oxide by-product.

22. The method of claim 21, wherein the compound comprising a halide or tosyl bound to a deprotonable carbon is methyl bromoacetate and the base is a carbonate.

23. The method of claim 21, further comprising precipitating the phosphine oxide by-product by adding a different solvent to the product mixture.

24. A method for converting an aldehyde functionality into an acetal functionality, the method comprising the step of reacting a substrate bearing an aldehyde functionality with a phosphonium salt of Formula I: 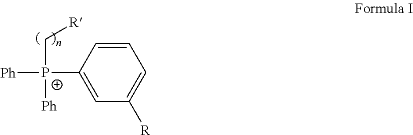
wherein:
n is 0 or 1;
R is H or SO₃⁻;
R′ is a C₁-C₁₀ alkoxycarbonyl when n is 1;
R′ is CX₃ when n is 0; and
X is selected from the group consisting of F, Cl, Br and I;
in the presence of an alcohol.

25. The method of claim 24, further comprising separating acetal product by adding to product mixture that includes a reaction solvent a different solvent in which phosphonium salt is not soluble.

26. The method of claim 25, further comprising filtering and evaporating of filtrate to obtain the acetal product.

27. A kit for converting an aldehyde functionality into an alkene functionality, comprising a zwitterionic phosphonium salt of Formula I: 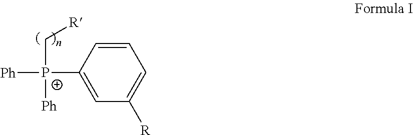
wherein:
n is 1;
R is SO₃⁻; and
R′ is selected from the group consisting of C₁-C₁₀ alkyl, C₂-C₁₀ alkenyl, C₂-C₁₀ alkynyl, C₃-C₁₀ cycloalkyl, phenyl, substituted phenyl, benzyl and C₁-C₁₀ alkoxycarbonyl; and
instructions for use.

28. A kit for converting an aldehyde functionality into an acetal functionality, comprising a phosphonium salt of Formula I: 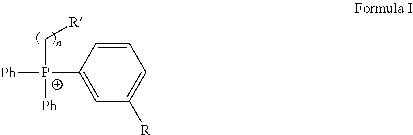
wherein:
n is 0;
R is H or SO₃⁻;
R′ is CX₃; and
X is selected from the group consisting of F, Cl, Br and I; and
instructions for use.

29. A kit for converting an aldehyde functionality into an alkene functionality comprising triphenylphosphine-meta-sulfonate, a compound comprising a halide or tosyl bound to a deprotonable carbon, and instructions for use.
